(19) United States
(12) Patent Application Publication
(10) Pub. No.: US 2015/0005747 A1
(43) Pub. Date: Jan. 1, 2015

(54) CAP AND TUBING APPARATUS AND RELATED METHODS

(71) Applicant: GENII, INC., St. Paul, MN (US)

(72) Inventors: Marcia Morris, St. Paul, MN (US); Duane Hiscock, St. Paul, MN (US)

(21) Appl. No.: 14/357,112

(22) PCT Filed: Nov. 8, 2012
(86) PCT No.: PCT/US12/64172

§ 371 Date: May 8, 2014

Related U.S. Application Data
(60) Provisional application No. 61/557,242, filed on Nov. 8, 2011.

(51) Int. Cl. A61B 1/00; A61B 1/12; A61M 3/02

(52) U.S. Cl. CPC A61B 1/00112; A61M 3/0287; A61B 1/00137; A61B 1/121
USPC 604/535

(57) ABSTRACT

An apparatus including a cap that can be placed onto or removed from a bottle with an interference fit, for instance using a snap-type motion. The cap has a top portion and at least partially open bottom portion, where the cap is defined in part by an outer top portion, outer side portion, inner top portion, and inner side portion. The top portion of the cap has a tubing interconnect, and the tubing interconnect includes a passage therethrough. The cap includes one or more venting passages.

### RELATED APPLICATION

This application claims priority to U.S. Provisional Patent Application No. 61/557,242, filed Nov. 8, 2011, the entirety of which is incorporated herein by reference.

### TECHNICAL FIELD

Bottle and bottle cap with tubing assemblies useful in lavage for flexible endoscopic and other medical procedures.

### SUMMARY

A tubing and cap apparatus attachable to a sterile or non-sterile water bottle for use with peristaltic lavage pump or flexible endoscope pressure systems which may include a peristaltic lavage pump for washing and lavage in flexible endoscopic and other medical procedures is described herein. The cap and tubing is used with a water bottle available commercially, or with a compatible disposable or reusable bottle intended to be used with the cap and tubing system. This apparatus can be used with stand-alone lavage pump systems or those attached to or associated with an electrosurgical system or other type of medical equipment. The subject apparatus includes a snap-on or push on cap having a top portion and an open, or partially open bottom portion, with the function of allowing the cap to be self-venting in that air can displace water inside the bottle during use. The cap is defined in part by an outer top portion, outer side portion, inner top portion, and inner side portion. The top or side portion of the cap may have a tubing passthrough or have a tubing interconnect, and the tubing interconnect includes a passage therethrough. The tubing interconnect may extend from a first tubing interconnect end portion to a second tubing interconnect end portion. In an option, the cap includes one or more projections of the cap that extend from the inner side portion. In one or more options, the cap includes at least one vent portion on a top portion of the cap, or at a location other than the top portion.

Several options for the apparatus are as follows. For example, in an option, the tubing interconnect has an outer tubing interconnect portion and/or an inner tubing interconnect portion, such as raised or recessed hubs, and optionally tubing is coupled with the outer tubing interconnect portion or the inner tubing interconnect portion. In another option the top has a passthrough opening for tubing to slide into and through.

In a further option, the internal projections within the cap are used for a friction or interference fit between the cap and bottle and are unilaterally spaced. In an option the friction or interference fit is provided by one or more internal projections within the cap. In another option, two or more projections include multiple projections including a first set and second set, the first set includes two or more projections disposed along a first half of the cap, and optionally the second set of projections has fewer projections than the first set of projections, or the second set of projections has a smaller collective length than a collective length of the first set of projections. In a further option, one or more venting passages are included within the cylindrical cap.

In another embodiment, a method includes pushing a push-on cap onto a bottle, the cap having a top portion and optionally an open or partially open_bottom portion, the cap defined in part by an outer top portion, outer side portion, inner top portion, and inner side portion, the top portion having a tubing interconnect, the tubing interconnect including a passage therethrough, where the cap is configured for used within a flexible endoscopy system. The method further includes optionally coupling a first tubing with an outer tubing interconnect of the cap, coupling a second tubing with an inner tubing interconnect of the cap, and venting gasses through venting passages of the cap. In an option, the method includes removing the cap from the bottle by pivoting the cap toward two or more projections within the cap and/or in effect ‘snapping’ on and off the cap from the bottle with an interference or friction fit.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE FIGURES

FIG. 1 is a side view of an apparatus according to one or more embodiments.

FIG. 2 is a bottom view of a bottle cap according to one or more embodiments.

FIG. 3 is a cross-sectional view of the bottle cap of FIG. 2 according to one or more embodiments taken along 3-3.

FIG. 4 is a bottom view of a bottle cap according to one or more embodiments.

FIG. 5 is a cross-sectional view of the bottle cap of FIG. 4 according to one or more embodiments taken along 5-5.

FIG. 6 is a side view of tubing according to one or more embodiments.

### DETAILED DESCRIPTION

The following detailed description includes references to the accompanying drawings, which form a part of the detailed description. The drawings show, by way of illustration, specific embodiments in which the apparatus may be practiced. These embodiments, which are also referred to herein as “examples” or “options,” are described in enough detail to enable those skilled in the art to practice the present embodiments. The embodiments may be combined, other embodiments may be utilized or structural or logical changes may be made without departing from the scope of the invention. The following detailed description is, therefore, not to be taken in a limiting sense and the scope of the invention is defined by the appended claims and their legal equivalents.

In this document, the terms “a” or “an” are used to include one or more than one, and the term “or” is used to refer to a nonexclusive “or” unless otherwise indicated. In addition, it is to be understood that the phraseology or terminology employed herein, and not otherwise defined, is for the purpose of description only and not of limitation.

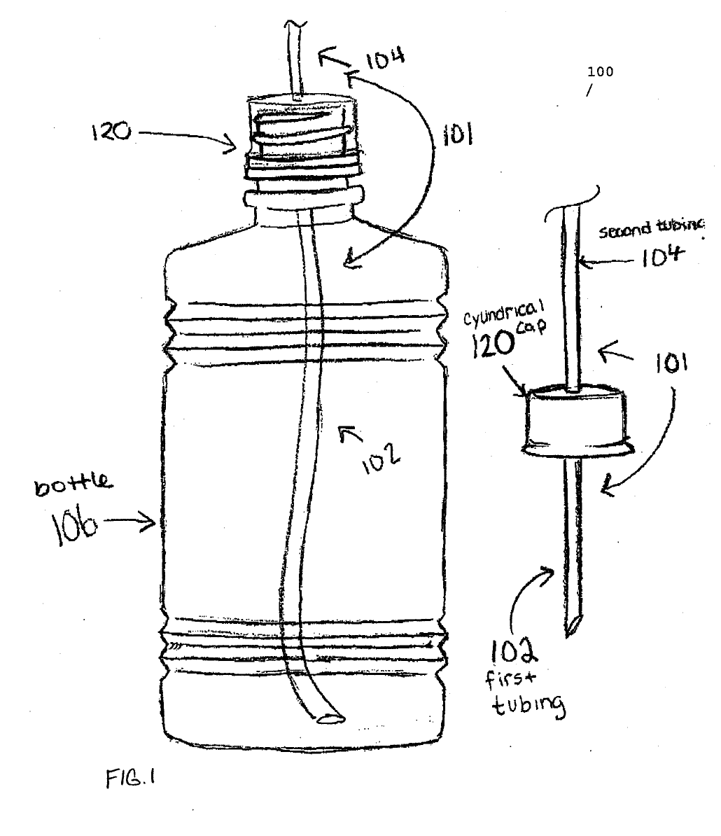

Referring to FIG. 1, an apparatus 100 such as a bottle and bottle cap assembly is illustrated. The apparatus 100 is configured to be attachable to a sterile or non-sterile water bottle for use with peristaltic lavage pump or flexible endoscope pressure systems which may include a peristaltic lavage pump for washing and lavage in flexible endoscopic and other medical procedures. The apparatus 100 includes tubing 101, such as a first tubing 102 and a second tubing 104, and a cap 120 coupled with a bottle 106. In one or more embodiments, the first and second tubing 102, 104 can be a single tube. The cap 120 can have a cylindrical, square, rectangular, or triangular shape. The bottle 106, in one or more embodiments, is a water source for use with a flexible endoscopy system, which may include a lavage pump, and includes an opening, and an optionally threaded portion. In a further option, the bottle 106 further includes one or more external projections for coupling with the cap 120. This bottle may be a standard, commercially available sterile water bottle, or may be one made specifically for use with a cap tubing apparatus, and can be filled, unfilled, or pre-filled.

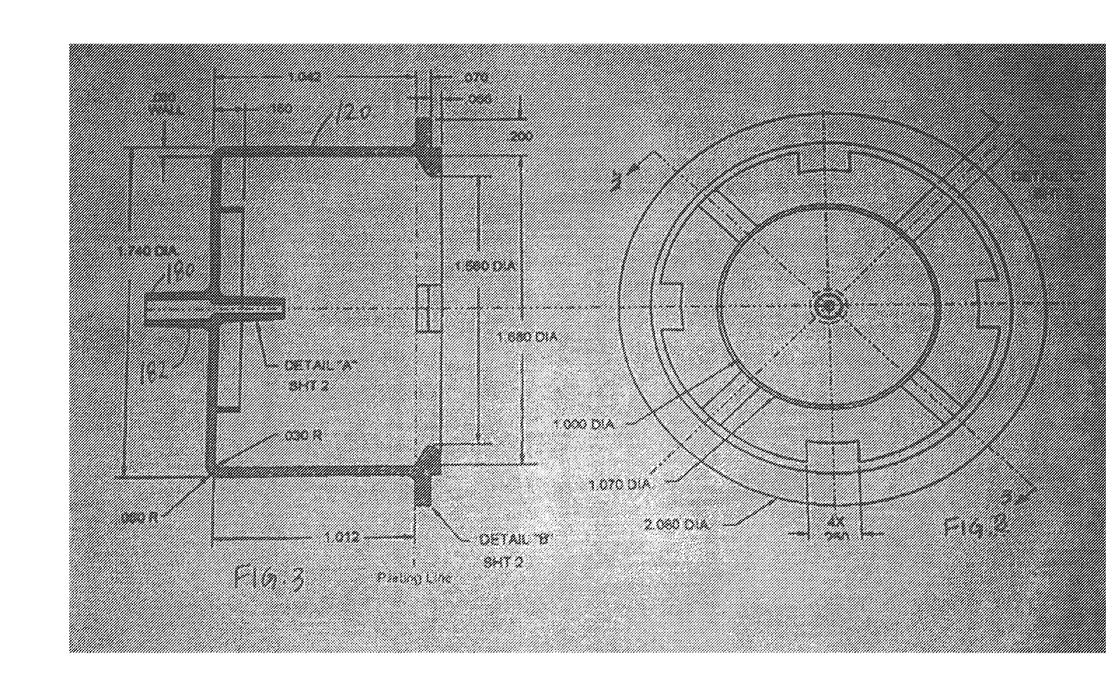

The cap 120 is shown in greater detail in FIGS. 2-5. In one or more embodiments, the cap is a flexible endoscopy system water source cap including at least one of a push on cap or a snap on cap. The cap 120 is defined in part by a top portion 122 and an open bottom portion 124, an outer top portion 126, an outer side portion 128, an inner top portion 130, and an inner side portion 132. In one or more embodiments, the open bottom portion 124 is at least partially open.

The cap 120 is a push-on cap. For example, the cap is couplable with the bottle 106 by pushing it on, for example with an interference fit or a snap-fit assembly, or a softer cap, i.e. the cap is formed of a material softer than the bottle. In an option, the cap 120 includes one or more, or two or more projections 150 that can couple with a portion of the bottle 106, for example by a ridge, flange or outer threads of the bottle 106. In an option, the projections 150 are disposed along an inner portion of the cap 120, for example extending from the inner side portion 132 of the cap 120 near the open bottom portion 124.

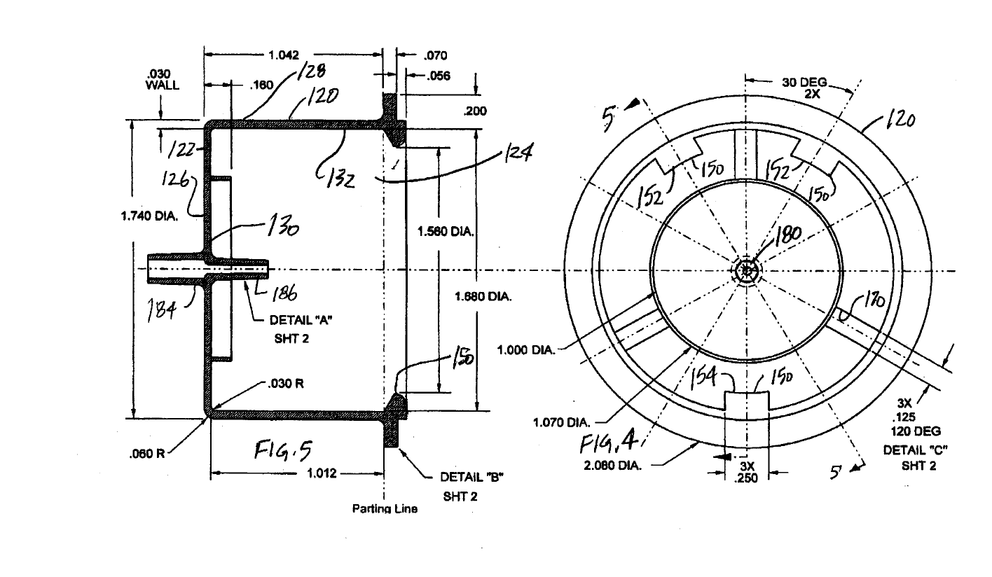

In one or more embodiments, the two or more projections 150 are three or more projections 150 spaced within the inner side portion 132. The three or more projections 150 are, in an option, unilaterally spaced within the cap 120, as shown in FIG. 2. In another option, the projections 150 are not unilaterally spaced within the cap 120, as shown in FIG. 4.

In a further option, the projections within the cap 120 include a first set of projections 152 and a second set of projections 154. In an option, the first set of projections 152 include two or more projections 150 disposed along a first half of the cap 120. In another option, the second set of projections 154 has fewer projections than the first set of projections 152. The set which has greater number of projections provides greater resistance to removing the cap 120 from the bottle, allowing for the cap 120 to be removed with a single handed operation, for example in a pivoting action.

In another option, the projections have an overall length that is defined by the amount of contact with the ridge, flange or threads of the bottle. In an option, the second set of projections 154 has a smaller collective length than a collective length of the first set of projections 152. The set which has smaller collective length provides lesser resistance to removing the cap 120 from the bottle, allowing for the cap 120 to be removed with a single handed operation, for example in a pivoting action.

In yet another option, the projections 150 can have a variety of profiles and/or number of projections and/or amount of interference with ridges, flanges or threads of the bottle. The projections are disposed along the cap such that the cap can be seated on the bottle and the projections extend under ridges, flanges or external threads of the bottle, relative to a top of the bottle, for example when the bottle is disposed in an upright position.

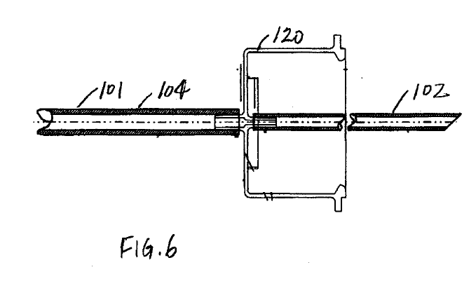

The apparatus 100, in another option, includes the cap 120 having a tubing interconnect 180. The tubing interconnect 180 includes a passage therethrough allowing for liquids or gasses (fluids) in the bottle to travel through the interconnect into the tubing which is optionally coupled with the interconnect 180. In an option, the tubing interconnect 180 includes a raised hub 182 that extends from the top portion of the cap 120 and forms an outer tubing interconnect portion 184. In another option, the interconnect 180 includes a raised hub, such as a second inner raised hub that extends toward the bottle when the cap is disposed on the bottle 106. The second inner raised hub forms an inner tubing interconnect portion 186. The tubing interconnect extends from a first tubing interconnect end portion to a second tubing interconnect end portion to allow fluids to pass therethrough. In an option, the tubing interconnect is an opening within the cap or a passage in the cap and the tubing passes through the opening or passage. In a further option, tubing 101 is coupled with the outer tubing interconnect portion 184, and/or tubing 101 is coupled with the inner tubing interconnect portion 186, as shown in FIG. 6. In an option, the tubing 101 coupled with the inner tubing interconnect portion 186 has a coupling disposed at a distal end portion, allowing for the tubing 101 within the bottle to be optionally weighted down toward a bottom portion of the bottle, assisting in delivery of substantially all of the fluid within the bottle. In another option, a length of the tubing 101 from the inner tubing interconnect portion 186 is longer than a length of the bottle, such that a portion of the length drapes along a bottom portion of the bottle when the tubing 101 is installed with the tubing interconnect of the cap, and the cap is installed on the bottle. The distal end of the 102 tubing may be cut at a slant to promote fluid flow and need not have any type of attached weight.

In yet another option, the cap includes at least one venting feature, such as one or more venting passages 170 within the cylindrical cap, as shown in FIGS. 2 and 4. The one or more venting passages 170 are, in an option, disposed between each of the projections. In an option, one or more ridges are formed on an inner portion of the cap. In an option the cap, when placed on the bottle, is spaced from the bottle, allowing for venting to occur. In an option, the venting occurs at a location other than a top portion of the cap, thereby eliminating a need for additional filtering or a membrane to be placed within the vent. In a further option, the venting can occur from side portions of the cap. In an option, the venting occurs at a location on the top portion of the cap. For instance, a vent can be formed in a top portion of the cap. In an option, a filter or membrane is positioned within the vent. In one or more embodiments, the venting feature is at a location other than a top of the cap.

In another embodiment, a method includes disposing a cap on a bottle, where the cap has a top portion and optionally an open or partially open bottom portion, and the cap is defined in part by an outer top portion, outer side portion, inner top portion, and inner side portion. The cap can include the various cap and cap options as discussed above and below. The cap and/or bottle are configured for use within a flexible endoscopy system. The cap optionally includes one or more projections extending from the inner side portion, for example, near the open bottom portion. The top portion of the cap has a tubing interconnect, and the tubing interconnect includes a passage therethrough. The tubing interconnect extends from a first tubing interconnect end portion to a second tubing interconnect end portion. The tubing can be a single piece of tubing disposed through the cap, or two or more sets of discrete tubing lengths coupled to hubs of the first tubing interconnect and/or a hub of the second tubing interconnect. Hubs can be projecting or recessed relative to the cap. The method further includes coupling a first tubing with an outer tubing interconnect of the cap, coupling a second tubing with an inner tubing interconnect of the cap, and venting gasses through venting passages of the cap. In an option, the method further includes removing the cap from the bottle by pivoting the cap toward one or more projections within the cap. In yet another option, the method further includes fastening or removing the cap via an interference fit.

In an option, coupling tubing with the cap includes coupling a first tubing with an outer tubing interconnect of the cap and coupling a second tubing with an inner tubing interconnect of the cap. In another option, coupling tubing with the cap includes disposing tubing through the cap.

The snap on cap is for use with a flexible endoscopy system, and is configured to be coupled with a water source. The water can be drawn from the water source, for example, with a lavage pump or with pressure from the endoscope. The snap on cap allows for ease of use and quick interchange of water supply for the endoscopy system.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope should, therefore, be determined with reference to the appended claims, along with the full scope of equivalents to which such claims are entitled.

1. An apparatus configured for use within a flexible endoscopy system, the apparatus comprising:
a flexible endoscopy system water source cap including at least one of a push-on or snap-on cap having a top portion, and at least a partially open bottom portion;
the cap defined in part by an outer top portion, outer side portion, inner top portion, and inner side portion;
the top portion having a tubing interconnect, the tubing interconnect including a passage therethrough;
the cap having at least one venting feature; and
the cap configured to be coupled with a water source.

2. The apparatus as recited in claim 1, wherein the at least one venting feature is within an inner portion of the cap.

3. The apparatus as recited in claim 1, wherein the at least one venting feature is at the outer top portion and/or inner top portion of the cap.

4. The apparatus as recited in claim 1, wherein the at least one venting feature is at a location other than the top portion of the cap.

5. The apparatus as recited in claim 1, wherein the tubing interconnect has at least one of an outer tubing interconnect portion or an inner tubing interconnect portion, and wherein the tubing interconnect portion includes at least one of a raised or recessed hub.

6. The apparatus as recited in claim 1, wherein the tubing interconnect extends from a first tubing interconnect end portion to a second tubing interconnect end portion.

7. The apparatus as recited in claim 5, further comprising tubing coupled with at least one of the outer tubing interconnect portion or the inner tubing interconnect portion.

8. The apparatus as recited in claim 1, further comprising a bottle, wherein the push-on or snap-on cap is coupled with the bottle with a snap-fit connection.

9. The apparatus as recited in claim 1, wherein the tubing interconnect is a hub.

10. The apparatus as recited in claim 1, wherein the at least one venting feature includes at least one of projections or ridges spaced within the inner side portion.

11. The apparatus as recited in claim 10, wherein the projections are unilaterally spaced.

12. The apparatus as recited in claim 1, wherein the at least one venting feature includes two or more projections having a first set and second set of projections, wherein the first set includes two or more projections disposed along a first half of the cap.

13. The apparatus as recited in claim 12, wherein the second set of projections has fewer projections than the first set of projections.

14. The apparatus as recited in claim 12, wherein the second set of projections has a smaller collective length than a collective length of the first set of projections.

15. A method comprising:
pushing at least one of a push-on or snap-on cap onto a bottle, the cap configured for use within a flexible endoscopy system, the cap having a top portion and an open bottom portion, the cap defined in part by an outer top portion, outer side portion, inner top portion, and inner side portion, the cap having a tubing interconnect, the tubing interconnect including a passage therethrough, the cap having one or more venting passages; and
venting gasses through one or more venting passages of the cap.

16. The method as recited in claim 15, further comprising coupling tubing with the cap, wherein coupling tubing with the cap includes coupling a first tubing with an outer tubing interconnect of the cap and coupling a second tubing with an inner tubing interconnect of the cap.

17. The method as recited in claim 15, further comprising coupling tubing with the cap, wherein coupling tubing with the cap includes disposing tubing through the cap.

18. The method as recited in claim 15, further comprising coupling tubing within recesses in the cap.

19. The method as recited in claim 15, further comprising removing the cap from the bottle by pivoting the cap toward or away from one or more projections within the cap.

20. The method as recited in claim 15, further comprising fastening or removing the cap via an interference fit.
